(19) United States
(12) Patent Application Publication
(10) Pub. No.: US 2012/0190740 A1
(43) Pub. Date: Jul. 26, 2012

(54) USE OF DRONEDARONE FOR THE PREPARATION OF A DRUG FOR USE IN THE MANAGEMENT OF THE RISK OF LIVER INJURY

(75) Inventor: Laurent AUCLERT, Paris (FR)

(73) Assignee: SANOFI, Paris (FR)

(21) Appl. No.: 13/323,141

(22) Filed: Dec. 12, 2011

Related U.S. Application Data
(60) Provisional application No. 61/421,798, filed on Dec. 10, 2010.

(30) Foreign Application Priority Data
Dec. 24, 2010 (EP) 10306511.6
Dec. 24, 2010 (EP) 10306514.0
Dec. 24, 2010 (EP) 10306516.5
Jan. 14, 2011 (EP) 11305037.1

(51) Int. Cl. A61K 31/343; A61P 9/06; A61P 9/00; A61P 1/16

(52) U.S. Cl. 514/469

(57) ABSTRACT

The present disclosure concerns methods of managing the risk of liver injury in patients receiving treatment with dronedarone or pharmaceutically acceptable salts thereof.

The present invention relates to method of using dronedarone for the preparation of a drug for use in the management of the risk of liver injury.

The present invention relates to methods of using dronedarone in a safe way in patients with cardiovascular history.

The instant invention also relates to a method of managing the risk of liver injury in patients receiving treatment with dronedarone or pharmaceutically acceptable salts thereof.

The instant invention also relates to a method of reducing the risk of liver injury in patients receiving treatment with dronedarone or pharmaceutically acceptable salts thereof.

2-n-Butyl-3-[4-(3-di-n-butylaminopropoxy)benzoyl]-5-methylsulphonamidobenzofuran, or dronedarone, and pharmaceutically acceptable salts thereof, in particular its hydrochloride salts, are described in European Patent EP 0 471 609 B1.

Moreover, dronedarone is effective in maintaining sinus rhythm in patients presenting atrial fibrillation or atrial flutter.

The applicant has clinically proven that dronedarone significantly reduces cardiovascular hospitalizations and/or mortality in patients having a history of atrial fibrillation (AF) or of atrial flutter (AFL) in a safe and effective way. In USA, Dronedarone is indicated to reduce the risk of cardiovascular hospitalization in patients with paroxysmal or persistent AF or AFL, with a recent episode of AF/AFL and associated cardiovascular risk factors (i.e., age >70, hypertension, diabetes, prior cerebrovascular accident, left atrial diameter 50 mm or left ventricular ejection fraction [LVEF]<40%), who are in sinus rhythm or who will be cardioverted.

The Applicant has now found the regimen to administrate dronedarone to patients in a safe and effective way, with a method to manage the risk of liver injury in patients receiving treatment with dronedarone or pharmaceutically acceptable salts thereof by performing the following steps:
a) performing liver function tests prior to treatment with dronedarone,
b) monitoring liver function monthly for six months, at months 9 and 12, and periodically thereafter,
c) if alanine aminotransferase (ALT) levels are elevated higher than three times the upper limit of normal (ULN), re-measuring levels,
d) if confirmed to be greater than three times the upper limit of normal, withdrawing administration of dronedarone,
e) continuing close observation until normalization of ALT and,
f) investigating the probable cause, including those related to underlying cardiac conditions.

The invention also relates to the regimen to administrate dronedarone to patients in a safe and effective way, with a method to manage the risk of liver injury in patients receiving treatment with dronedarone or pharmaceutically acceptable salts thereof by performing the following steps:
a) performing liver function tests prior to treatment with dronedarone,
b) monitoring liver function monthly for six months, at months 9 and 12, and periodically thereafter,
c) if alanine aminotransferase (ALT) levels are elevated higher than three times the upper limit of normal (ULN), re-measuring levels for example within 48 to 72 hours,
d) if confirmed to be greater than three times the upper limit of normal, withdrawing administration of dronedarone,
e) continuing close observation until normalization of ALT and,
f) investigating the probable cause, including those related to underlying cardiac conditions.

Mention may be made that “performing liver function tests prior to treatment with dronedarone” means obtaining a blood sample from the patient prior to treatment with dronedarone then performing liver function tests prior to treatment with dronedarone.

Mention may be made that “monitoring liver function monthly for six months, at months 9 and 12, and periodically thereafter” means “obtaining a blood sample from the patient in order to perform liver function tests and monitoring liver function after initiation of dronedarone administration, monthly for six months, at months 9 and 12, and periodically thereafter”.

Thus, the invention also relates to the regimen to administrate dronedarone to patients in a safe and effective way, with a method to manage the risk of liver injury in patients receiving treatment with dronedarone or pharmaceutically acceptable salts thereof by performing the following steps:
a) obtaining a blood sample from the patient prior to treatment with dronedarone,
b) performing liver function tests prior to treatment with dronedarone,
c) initiating dronedarone administration,
d) obtaining a blood sample from the patient in order to perform liver function tests and monitoring liver function after initiation of dronedarone administration, monthly for six months, at months 9 and 12, and periodically thereafter,
   and additionally by performing the following steps:
e) if alanine aminotransferase (ALT) levels are elevated higher than three times the upper limit of normal (ULN), re-measuring levels,
f) if confirmed to be greater than three times the upper limit of normal, withdrawing administration of dronedarone,
g) continuing close observation until normalization of ALT,
h) investigating the probable cause, including those related to underlying cardiac conditions.

According to one embodiment, step d) is performed one week, one month, monthly for six months, at months 9 and 12, and periodically after initiation of dronedarone administration.

The invention also relates to dronedarone or one of its pharmaceutically acceptable salt for use in a safe way in patients with a cardiovascular history, particularly in patients with Atrial fibrillation or flutter, more particularly in patients with paroxysmal or persistent atrial fibrillation, said use comprising the following steps:
a) obtaining a blood sample from the patient prior to treatment with dronedarone,
b) performing liver function tests prior to treatment with dronedarone,
c) initiating dronedarone administration, and
d) obtaining a blood sample from the patient in order to perform liver function tests and monitoring liver function after initiation of dronedarone administration, monthly for six months, at months 9 and 12, and periodically thereafter.

The invention also relates to dronedarone or one of its pharmaceutically acceptable salt for use in a safe way in patients with a cardiovascular history, particularly in patients with Atrial fibrillation or flutter, more particularly in patients with paroxysmal or persistent atrial fibrillation, said use comprising the following steps:
a) obtaining a blood sample from the patient prior to treatment with dronedarone,
b) performing liver function tests prior to treatment with dronedarone,
c) initiating dronedarone administration, and
d) obtaining a blood sample from the patient in order to perform liver function tests and monitoring liver function after one week and after one month following initiation of dronedarone administration initiation, monthly for six months, at months 9 and 12, and periodically thereafter.

Additionally the use according to the invention comprises the above mentioned steps e) to h).

The invention also relates to a method of treating arrhythmia in a patient comprising:
a) initiating treatment with dronedarone, or a pharmaceutically acceptable salt thereof, in a patient suffering from arrhythmia; and
b) discontinuing treatment with dronedarone, or a pharmaceutically acceptable salt thereof, if liver injury in said patent is suspected.

According to one embodiment, the patient's liver enzymes are obtained during the first 6 months after initiating treatment with dronedarone or pharmaceutically acceptable salt thereof. In one embodiment, the patient's liver enzymes monthly during the first 6 months, after initiating treatment with dronedarone or pharmaceutically acceptable salt thereof. In another embodiment, the patient's liver enzymes at about 9 months and about 12 months after initiating treatment with dronedarone or pharmaceutically acceptable salt thereof.

This invention also relates to a method of treating arrhythmia in a patient comprising:
a) initiating treatment with dronedarone, or a pharmaceutically acceptable salt thereof, in a patient suffering from arrhythmia, wherein said patient's liver enzymes were measured prior to initiating treatment; and
b) discontinuing treatment with dronedarone, or a pharmaceutically acceptable salt thereof, in a patient whose alanine aminotransferase (ALT) levels are elevated higher than three times the upper limit of normal (ULN) in a measurement of the patient's liver enzymes after treatment initiation.

Liver injury may comprise hepatic events such as liver function test abnormalities and hepatocellular liver injury, including acute hepatic failure or life-threatening acute liver failure.

Liver functions test may comprise determination of liver enzymes levels. These enzymes may be alkaline phosphatase (ALP, AP), alanine aminotransferase (ALT), aspartate aminotransferase (AST), total bilirubin.

Symptoms in patients which suggest hepatic injury comprise anorexia, vomiting, fever, malaise, fatigue, right upper quadrant pain, jaundice, dark urine and itching.

“Managing the risk” may be defined as “reducing the risk”.

The uses and methods according to the invention enable to decrease the risk of liver injury, when dronedarone or pharmaceutically acceptable salts or esters thereof is administered for treating patients with paroxysmal or persistent atrial fibrillation (AF) or atrial flutter (AFL), with a recent episode of AF/AFL and associated cardiovascular risk factors (i.e., age >70, hypertension, diabetes, prior cerebrovascular accident, left atrial diameter 50 mm or left ventricular ejection fraction [LVEF]<40%), who are in sinus rhythm or who will be cardioverted.

The patients concerned by the present invention have a cardiovascular history. They may be affected, for example, by a Atrial Fibrillation such as a persistent Atrial Fibrillation, a paroxysmal Atrial Fibrillation or a permanent Atrial Fibrillation or by a Atrial Flutter.

According to another embodiment, patients are chosen from patients with paroxysmal or persistent atrial fibrillation.

The American College of Cardiology, American Heart Association, and the European Society of Cardiology recommend in their guidelines the following classification system based on simplicity and clinical relevance:
Patients with Paroxysmal Atrial Fibrillation means patients with recurrent episodes that self-terminate in less than 7 days.)
Patients with persistent Atrial Fibrillation means patients with recurrent episodes that last more than 7 days.

If a first detected episode self-terminates in less than 7 days and then another episode begins later on, the case has moved into the category of paroxysmal AF. Although patients in this category have episodes lasting up to 7 days, in most cases of paroxysmal AF the episodes will self-terminate in less than 24 hours. If instead the episode lasts for more than 7 days, it is unlikely to self-terminate and it is called persistent AF. In this case, the episode may be terminated by cardioversion.)
If cardioversion is unsuccessful or it is not attempted, and the episode is ongoing for a long time (e.g. a year or more), the patient's AF is called permanent.

Among the patients having a cardiovascular history according to the invention, in particular having a history of atrial fibrillation or atrial flutter, mention may also be made of patients also exhibiting at least one additional risk factors, such as:
age equal to or above 70, or even above 75
hypertension,
diabetes,
prior cerebrovascular disease,
left atrial diameter greater than or equal to 50 mm measured by echocardiography,
left ventricular ejection fraction less than 40%, measured by two-dimensional echography.

Among the patients having a cardiovascular history according to the invention, in particular having a history of atrial fibrillation or atrial flutter, mention may also be made of patients also exhibiting additional risk factors, i.e. at least one pathologies chosen from:
Structural heart disease,
Lone Atrial Fibrillation,
Coronary heart disease, and
Dilated cardiomyopathy,
   and/or at least one cardiac device chosen from:
Pacemaker, and
Valvular heart.

Mention may be made that “method to manage the risk of liver injury” may be understood as
use of dronedarone or one of its pharmaceutically acceptable salt, for the preparation of a drug for use in the prevention of liver injury,
use of dronedarone or one of its pharmaceutically acceptable salt, for the preparation of a drug for use in the management of the risk of liver injury,
dronedarone or one of its pharmaceutically acceptable salt for use in the management of the risk of liver injury,
dronedarone or one of its pharmaceutically acceptable salt for use in the prevention of liver injury,
   and vice-versa.

Mention may be made that “dronedarone for use in” may be understood as use of dronedarone for the preparation of a medicament for use in “and vice-versa.

For their therapeutic use, dronedarone and pharmaceutically acceptable salts thereof are generally introduced into pharmaceutical compositions.

These pharmaceutical compositions contain an effective dose of dronedarone or of a pharmaceutically acceptable salt thereof, and also at least one pharmaceutically acceptable excipient.

Said excipients are chosen according to the pharmaceutical form and the method of administration desired, from the usual excipients which are known to those skilled in the art.

In said pharmaceutical compositions for oral, sublingual, subcutaneous, intramuscular, intravenous, topical, local, intratracheal, intranasal, transdermal or rectal administration, dronedarone, or the salt thereof, can be administered in unit administration form, as a mixture with conventional pharmaceutical excipients, to animals and to humans in the cases mentioned above.

The suitable unit administration forms comprise forms for oral administration, such as tablets, soft or hard gel capsules, powders, granules and oral solutions or suspensions, sublingual, buccal, intratracheal, intraocular or intranasal administration forms, forms for administration by inhalation, topical, transdermal, subcutaneous, intramuscular or intravenous administration forms, rectal administration forms, and implants. For topical application, dronedarone and pharmaceutically acceptable salts thereof can be used in creams, gels, ointments or lotions.

By way of example, a unit administration form of dronedarone or a pharmaceutically acceptable salt thereof, in tablet form, may correspond to one of the following compositions (Examples 1-4) according to the invention:

EXAMPLE 1

| Ingredients | mg |
|---|---|
| Dronedarone hydrochloride (corresponding to 400 mg of | 426 |
| base) | |
| Methylhydroxypropylcellulose | 21.1 |
| Lactose monohydrate | 46.55 |
| Maize starch | 45.5 |
| Polyvinylpyrrolidone | 65 |
| Poloxamer 407 | 40 |
| Anhydrous colloidal silica | 2.6 |
| Magnesium stearate | 3.25 |
| | 650 |

EXAMPLE 2

| Ingredients | mg |
|---|---|
| Dronedarone hydrochloride (corresponding to 400 mg of | 426 |
| base) | |
| Microcrystalline cellulose | 65 |
| Anhydrous colloidal silica | 2.6 |
| Anhydrous lactose | 42.65 |
| Polyvinylpyrrolidone | 13 |
| Poloxamer 407 | 40 |
| Macrogol 6000 | 57.5 |
| Magnesium stearate | 3.25 |
| | 650 |

EXAMPLE 3

| Ingredients | mg |
|---|---|
| Dronedarone hydrochloride (corresponding to 400 mg of | 426 |
| base) | |
| Microcrystalline cellulose | 26 |
| Maize starch | 45.5 |
| Polyvinylpyrrolidone | 65 |
| Poloxamer 407 | 40 |
| Anhydrous colloidal silica | 3.25 |
| Magnesium stearate | 3.25 |
| Lactose monohydrate | 41.65 |
| | 650 |

EXAMPLE 4

| Ingredients | mg |
|---|---|
| Dronedarone hydrochloride (corresponding to 200 mg of | 213 |
| base) | |
| Microcrystalline cellulose | 13 |
| Maize starch | 22.75 |
| Polyvinylpyrrolidone | 32.5 |
| Poloxamer 407 | 20 |
| Anhydrous colloidal silica | 1.3 |
| Magnesium stearate | 1.625 |
| Lactose monohydrate | 20.825 |
| | 325 |

The dose of dronedarone administered per day, orally, may reach 800 mg, taken in one or more intakes. More specifically, the dose of dronedarone administered may be taken with food. For example, the dose of dronedarone administered per day, orally, may reach 800 mg, taken in two intakes with a meal.

The dose of dronedarone administered per day, orally may be taken at a rate of twice a day (usually abbreviated BID) with a meal for example with the morning and the evening meal. More specifically, the two intakes may comprise same quantity of dronedarone.

Advantageously, the dose of dronedarone administered, orally, may reach 400 mg BID, taken together with a meal, for example with the morning and the evening meal.

There may be specific cases where higher or lower dosages are appropriate; such dosages do not depart from the context of the invention. According to the usual practice, the dosage appropriate for each patient is determined by the physician according to the method of administration, the weight, the pathology, the body surface, the cardiac output and the response of said patient.

According to another of its aspects, the present invention also relates to a method for treating the pathologies indicated above, which comprises the administration, to a patient, of an effective dose of dronedarone or a pharmaceutically acceptable salt thereof.

### BRIEF DESCRIPTION OF THE DRAWINGS

The present invention is illustrated by the data hereinafter with reference to the attached drawings in which:

FIG. 1 represents a Kaplan Meier curve with the cumulative rate of first hepatic event or ALT greater than or equal to 5 times ULN during the on-treatment period.

In the pool of 5 placebo controlled studies in patients with AF/AFL (DR13550/DAFNE, EFC4508/ERATO, EFC3153/EURIDIS, EFC4788/ADONIS, EFC5555/ATHENA), the hepatic events were analyzed with the following selection:
ALT 5×ULN, or
SOC ‘Hepatobiliary disorders’, or
SMQ “Liver related investigations signs and symptoms” using broad and narrow selection

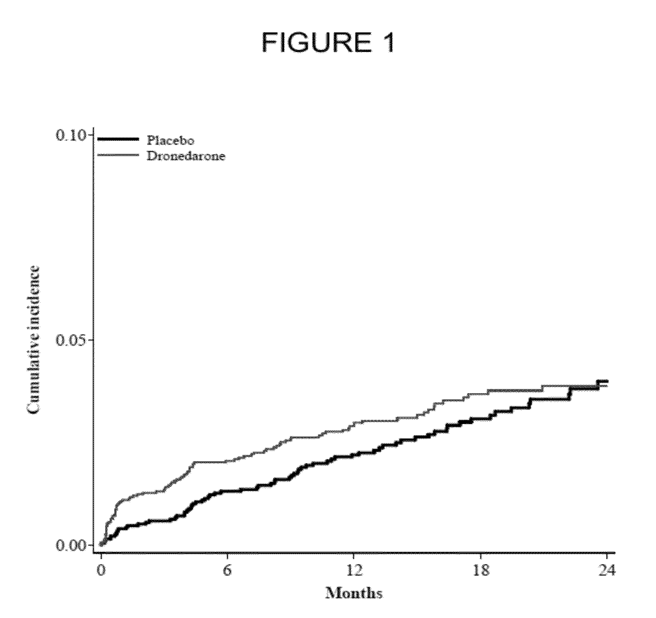

The time to onset analysis of these hepatic events showed a trend in which the dronedarone group had an earlier onset of hepatic events during the first 6 months compared to placebo (FIG. 1).

After 1 year, the incidences appeared to be similar in the 2 groups, the data being entirely driven by adverse events reported in the ATHENA study (enzymes not routinely collected).

In addition, two spontaneously post marketing safety reports of hepatic failure leading to liver transplantation were reported in patients treated with MULTAQ® (dronedarone).
CASE 1: A 69-year-old female patient with normal baseline liver function tests, experienced acute hepatic failure 4.5 months after starting treatment with dronedarone for recurrent re-entry tachycardia (off label indication) and 7 days after her last dose of dronedarone (reason for drug withdrawal not stated). She required a liver transplant and was still in intensive care at the time of the last report. Concomitant medications included lisinopril, hydrochlorothiazide, bisoprolol, amlodipine, levothyroxine, simvastatin, acetylsalicylic acid, alendronic acid, tiotropium bromide and formoterol. Relevant history included triple vessel disease coronary artery disease, COPD, skin tumor and heterozygous Factor V Leiden mutation. Histological examination of the explanted liver was reported as consistent with a drug-induced toxicity.
CASE 2: A 72-year-old female patient with medical history of paroxysmal atrial fibrillation and Sjögren's syndrome experienced acute hepatic failure almost six months after starting dronedarone. She underwent successful liver transplantation. At the time of the report the patient was recovering and was still in the intensive care unit. Liver function tests 3 months prior to dronedarone start had been within normal limits. Concomitant medications included metoprolol, amlodipine, omeprazole, warfarin, alprazolam, calcium, biotin and multivitamins. No evidence for autoimmune hepatitis was found. The liver histology revealed 60% to 70% necrosis. All details of the histopathology evaluation were not available at time of report.

Thus, those events occurred within 6 months after dronedarone treatment initiation and consequently support the claimed invention to monitor liver function and risk of liver injury.

What is claimed is:

1. A method of safely using dronedarone or one of its pharmaceutically acceptable salts in a patient with a cardiovascular history, said use comprising the following steps:
a) obtaining a blood sample from the patient prior to treatment with dronedarone,
b) performing liver function tests prior to treatment with dronedarone,
c) initiating dronedarone administration, and
d) obtaining a blood sample from the patient in order to perform liver function tests and monitoring liver function after initiation of dronedarone administration, monthly for six months, at months 9 and 12, and periodically thereafter.

2. The method according to claim 1 wherein said method additionally comprises the following steps e) to h):
e) if alanine aminotransferase (ALT) levels are elevated higher than three times the upper limit of normal (ULN), re-measuring levels,
f) if confirmed to be greater than three times the upper limit of normal, withdrawing administration of dronedarone,
g) continuing close observation until normalization of ALT, and
h) investigating the probable cause, including those related to underlying cardiac conditions.

3. The method according to claim 2 wherein the re-measuring levels is performed within 48 to 72 hours.

4. The method according to claim 1 wherein step d) is performed one week, one month, monthly for six months, at months 9 and 12, and periodically after initiation of dronedarone administration.

5. The method according to claim 1 wherein liver injury comprises hepatocellular liver injury.

6. The method according to claim 1 wherein the liver injury comprises life-threatening acute liver failure.

7. The method according to claim 1 wherein liver functions test comprise determination of liver enzymes levels.

8. The method according to claim 1 wherein the patient has persistent Atrial Fibrillation.

9. The method according to claim 1 wherein the patient has paroxysmal Atrial Fibrillation.

10. The method according to claim 1 wherein the patient has permanent Atrial Fibrillation.

11. The method according to claim 1 wherein the dose of dronedarone administered orally is 400 mg BID.

12. The method according to claim 1 wherein the patient also exhibits at least one additional cardiovascular risk factor chosen from:
age equal to or above 70, or even above 75,
hypertension,
diabetes,
prior cerebrovascular disease,
left atrial diameter greater than or equal to 50 mm measured by echocardiography, and
left ventricular ejection fraction less than 40%, measured by two-dimensional echography.

13. The method according to claim 1, wherein the patient also exhibits at least one pathology chosen from:
Structural heart disease,
Lone Atrial Fibrillation,
Coronary heart disease, and
Dilated cardiomyopathy,
and/or at least one cardiac device chosen from:
Pacemaker, and
Valvular heart.

14. A method of managing the risk of liver injury in a patient receiving treatment with dronedarone or a pharmaceutically acceptable salt thereof, said method comprising the following steps:
a) obtaining a blood sample from the patient prior to treatment with dronedarone,
b) performing liver function tests prior to treatment with dronedarone,
c) initiating dronedarone administration, and
d) obtaining a blood sample from the patient in order to perform liver function tests and monitoring liver function after initiation of dronedarone administration, monthly for six months, at months 9 and 12, and periodically thereafter.

15. The method according to claim 14 wherein said method additionally comprises the following steps e) to h):
e) if alanine aminotransferase (ALT) levels are elevated higher than three times the upper limit of normal (ULN), re-measuring levels,
f) if confirmed to be greater than three times the upper limit of normal, withdrawing administration of dronedarone,
g) continuing close observation until normalization of ALT and,
h) investigating the probable cause, including those related to underlying cardiac conditions.

16. A method of treating arrhythmia in a patient comprising:
a) initiating treatment with dronedarone, or a pharmaceutically acceptable salt thereof, in a patient suffering from arrhythmia; and
b) discontinuing treatment with dronedarone, or a pharmaceutically acceptable salt thereof, if liver injury in said patent is suspected.

17. The method according to claim 16 wherein the patient has persistent Atrial Fibrillation.

18. The method according to claim 16 wherein the patient has paroxysmal Atrial Fibrillation.

19. The method according to claim 16 wherein the patient has permanent Atrial Fibrillation.

20. The method according to claim 16 wherein the dose of dronedarone administered orally, is 400 mg BID.

21. The method according to claim 16 wherein the patient also exhibits at least one additional cardiovascular risk factor chosen from:
age equal to or above 70, or even above 75,
hypertension,
diabetes,
prior cerebrovascular disease,
left atrial diameter greater than or equal to 50 mm measured by echocardiography, and
left ventricular ejection fraction less than 40%, measured by two-dimensional echography.

22. The method according to claim 16, wherein the patient also exhibits at least one pathologies chosen from:
Structural heart disease,
Lone Atrial Fibrillation,
Coronary heart disease, and
Dilated cardiomyopathy,
and/or at least one cardiac device chosen from:
Pacemaker, and
Valvular heart.

23. The method according to claim 16, wherein the liver injury comprises hepatocellular liver injury.

24. The method according to claim 16 further comprising obtaining the patient's liver enzymes during the first 6 months after initiating treatment with dronedarone or pharmaceutically acceptable salt thereof.

25. The method according to claim 16 further comprising obtaining the patient's liver enzymes monthly during the first 6 months after initiating treatment with dronedarone or pharmaceutically acceptable salt thereof.

26. The method according to claim 25 further comprising obtaining the patient's liver enzymes at about 9 months and about 12 months after initiating treatment with dronedarone or pharmaceutically acceptable salt thereof.

27. The method according to claim 16 wherein liver injury is suspected in said patient from the measurement of one or more values selected from aspartate aminotransferase, alanine aminotransferase, alkaline phosphatase and serum bilirubin.

28. The method according to claim 16, wherein the patient with suspected liver injury exhibits one or more symptoms selected from the group consisting of anorexia, vomiting, fever, malaise, fatigue, right upper quadrant pain, jaundice, dark urine and itching.

29. A method of treating arrhythmia in a patient comprising:
a) initiating treatment with dronedarone, or a pharmaceutically acceptable salt thereof, in a patient suffering from arrhythmia, wherein said patient's liver enzymes were measured prior to initiating treatment; and
b) discontinuing treatment with dronedarone, or a pharmaceutically acceptable salt thereof, in a patient whose alanine aminotransferase (ALT) levels are elevated higher than three times the upper limit of normal (ULN) in a measurement of the patient's liver enzymes after treatment initiation.
